(19) United States
(12) Patent Application Publication
(10) Pub. No.: US 2012/0142103 A1
(43) Pub. Date: Jun. 7, 2012

(54) METHOD FOR INDUCING DIFFERENTIATION INTO EPITHELIAL PROGENITOR CELL/STEM CELL POPULATION AND CORNEAL EPITHELIAL CELL POPULATION FROM INDUCED PLURIPOTENT STEM CELLS

(76) Inventors: Kohji Nishida, Miyagi (JP); Ryuhei Hayashi, Miyagi (JP); Miharu Sakurai, Miyagi (JP); Tomofumi Kageyama, Miyagi (JP); Shinya Yamanaka, Kyoto (JP); Keisuke Okita, Kyoto (JP)

(21) Appl. No.: 13/321,130

(22) PCT Filed: May 18, 2010
(86) PCT No.: PCT/JP2010/058689

§ 371 Date: Feb. 10, 2012

(51) Int. Cl. C12N 5/071

(52) U.S. Cl. 435/377

(57) ABSTRACT

The present invention relates to: a method for inducing differentiation into an epithelial progenitor cell/stem cell population or a corneal epithelial cell population by culturing, under particular conditions, induced pluripotent stem cells induced from mammalian somatic cells or undifferentiated stem cells; an epithelial progenitor cell/stem cell population or a corneal epithelial cell population obtained by the method; and a cell preparation for the treatment of epithelial disease and a cell sheet, which are prepared using these cell populations.

### TECHNICAL FIELD

The present invention relates to: a method for inducing differentiation into an epithelial progenitor cell/stem cell population or a corneal epithelial cell population from induced pluripotent stem cells induced from mammalian somatic cells or undifferentiated stem cells; and a use of a cell population induced by said method in the treatment of epithelial disease.

### BACKGROUND ART

Keratoplasty based on eye donation has been carried out for intractable corneal epithelial disease and, however, has the problems of absolute donor shortage and rejection after transplantation. To solve the problems, therapy has been developed using patient's own corneal limbus cells or oral mucosal epithelial cells. In this method, a cultured corneal epithelial cell sheet is prepared from corneal limbus cells of healthy eyes or oral mucosal epithelial cells and transplanted to an affected eye (Patent Literatures 1 and 2 and Non Patent Literature 1). However, the method using corneal limbus epithelial cells cannot be adapted to patients with disease in both eyes. Also, since the oral mucosal epithelium does not differentiate into complete corneal epithelium, this method has the risk of causing the invasion of blood after transplantation.

By contrast, research on regenerative medicine to compensate for injured tissues or organs by inducing the differentiation of undifferentiated cells (stem cells) has been developed. Embryonic stem cells (ES cells) can differentiate into all cells except placenta. Thus, the induction of their differentiation into each cell lineage or the identification of a determinant factor for the differentiation has drawn attention. However, the research or use of the ES cells is largely limited due to ethical problems. Also, these ES cells have the problem of rejection and thus, have not been clinically applied yet.

Recently, induced pluripotent stem cells that have the pluripotency similar to ES cells have been established by introducing the defined factors into somatic cells or undifferentiated stem cells. A typical example thereof is iPS cells that have been established by Yamanaka et al. (Patent Literature 3 and Non Patent Literatures 2 and 3). Regenerative medicine using these induced pluripotent stem cells is not only free from ethical problems but also can avoid the problem of rejection by using patient-derived cells as a source.

Meanwhile, human embryos form, at the developmental stage, three germ layers: endoderm, mesoderm, and ectoderm. The endoderm differentiates into gastric or small intestinal mucosal epithelium, the liver, the pancreas, and the like. The mesoderm differentiates into muscles, bones, blood vessels or blood, subcutaneous tissues, the heart, the kidney, and the like. The ectoderm forms nerves, eyes (corneal epithelium), the epidermis, and the like. In addition, the neural crest, which differentiates into peripheral nerves, glial cells, or some ganglia, is also called the fourth germ layer.

With respect to the induction of differentiation into ectodermal cells from ES cells, the induction of differentiation into epidermal cells and nerve cells has been previously reported. Specifically, Green and Haase et al. have reported that p63+ or keratin 14 (K14)+ epidermal cells were obtained by plate-culturing in a FAD medium embryoid bodies formed from ES cells or cells isolated from nodules obtained by administering ES cells to SCID mice (Patent Literature 4 and Non Patent Literatures 4 and 5). Moreover, Sasai and Mizuseki et al. have reported that nerve cells were induced from ES cells by using a method, called SDIA (Stromal cell-derived inducing activity) method, using mouse-derived stromal cells (PA6 cells) (Patent Literatures 5 and 6 and Non Patent Literatures 6 to 8).

However, none of the previous documents have specifically reported the induction of differentiation into epithelial cells such as corneal epithelial cells from ES cells or iPS cells.

### Citation List

### Patent Literature

Patent Literature 1: WO2004/069295

Patent Literature 2: Japanese Patent Laid-Open No. 2005-130838

Patent Literature 3: WO2007/069666

Patent Literature 4: WO2005/056765

Patent Literature 5: WO2001/088100

Patent Literature 6: WO2003/042384

### Non Patent Literature

Non Patent Literature 1: Nishida K et al., N. Engl. J. Med., (2004) 351: 1187-96

Non Patent Literature 2: Takahashi K, Yamanaka S., Cell, (2006) 126: 663-676

Non Patent Literature 3: Takahashi K, Yamanaka S., et al., Cell, (2007) 131: 861-872.

Non Patent Literature 4: Green H et al., Proc. Natl. Acad. Sci., USA, (2003) 15625-15630

Non Patent L*terature 5: Haase I et al., Eur. J. Cell Biol., (2007) 801-805

Non Patent Literature 6: Kawasaki, H., Sasai, Y. et al., Neuron, (2000) 28, 31-40.

Non Patent Literature 7: Kawasaki, H., Sasai, Y. et al., Proc. Natl. Acad. Sci., USA 99, (2002) 1580-1585

Non Patent Literature 8: Mizuseki, K., Sasai, Y. et al., Proc. Natl. Acad. Sci., USA 100, (2003) 5828-5833

### SUMMARY OF INVENTION

### Technical Problem

An object of the present invention is to solve the problems of donor shortage and rejection by developing epithelial stem cells/progenitor cells or corneal epithelial cells from patient's own cells and thereby providing novel means for the treatment of epithelial disease including keratoplasty.

### Solution to Problem

The present inventors have repeated experiments under various conditions for inducing differentiation into epithelial cells of interest from induced pluripotent stem cells (iPS cells) and, consequently, have successfully induced iPS cell-derived epithelial stem cells/progenitor cells having morphology and properties (p63-positive and keratin 14-positive) equivalent to those of epithelial stem cells/progenitor cells in vivo. The present inventors have further successfully induced corneal epithelial differentiation marker keratin 12-positive cells from the iPS cell-derived epithelial stem cells/progenitor cells.

If epithelial stem cells/progenitor cells and corneal epithelial cells can be developed by this method using induced pluripotent stem cells prepared from patient's own cells, the cornea can be regenerated without being concerned about the problems of donor shortage and rejection. The obtained corneal epithelial cells can be used as a layered-cultured corneal epithelial cell sheet by the method as described above to thereby provide more favorable corneal regeneration treatment.

Specifically, according to the first embodiment, the present invention provides a method for inducing differentiation into a keratin 14-positive and p63-positive epithelial progenitor cell/stem cell population from induced pluripotent stem cells induced from mammalian somatic cells or undifferentiated stem cells, comprising: culturing said induced pluripotent stem cells on feeder cells or a support selected from collagen (preferably type I or type IV collagen), basement membrane matrix, amnion, fibronectin, and laminin using a medium for epidermal cells containing an epidermal growth factor and/or cholera toxin and serum.

In said method, it is preferred that the medium should further contain one or more selected from hydrocortisone, insulin, transferrin, and selenium.

Moreover, it is preferred that the medium should further contain BMP4 (Bone Morphogenetic Protein 4). Furthermore, it is more preferred that the medium should further contain retinoic acid. In this context, the retinoic acid also includes salts or derivatives thereof usually used.

Examples of the feeder cells used can include, but not limited to, stromal cells such as 3T3 cells.

In said method, it is preferred that the induced pluripotent stem cells should be induced to differentiate into the epithelial progenitor cell/stem cell population without embryoid body formation.

According to the second embodiment, the present invention provides a method for inducing differentiation into a keratin 14-positive and p63-positive epithelial progenitor cell/stem cell population from induced pluripotent stem cells induced from mammalian somatic cells or undifferentiated stem cells, comprising: culturing said induced pluripotent stem cells on 3T3 cells or in the presence of a 3T3 cell-derived differentiation factor.

In the said method, the induced pluripotent stem cells are cultured in an epithelial induction medium containing serum and/or BMP4 or a medium for epidermal cells (e.g., a KCM medium) containing an epidermal growth factor and/or cholera toxin and serum. The epithelial induction medium may further contain one or more selected from retinoic acid, nonessential amino acid, β-mercaptoethanol, and sodium pyruvate. Moreover, the medium for epidermal cells may further contain one or more selected from hydrocortisone, insulin, transferrin, and selenium.

In said method, it is preferred that the induced pluripotent stem cells should be cultured in a differentiation medium containing a serum substitute such as KSR and/or BMP4 before being cultured in the epithelial induction medium or the medium for epidermal cells. It is more preferred that the differentiation medium should further contain retinoic acid. The differentiation medium may further contain one or more selected from nonessential amino acid, β-mercaptoethanol, and sodium pyruvate. As described above, the retinoic acid also includes salts or derivatives thereof usually used.

It is particularly preferred that the epithelial induction medium should further contain BMP4 (Bone Morphogenetic Protein 4).

According to the third embodiment, the present invention provides a method for inducing differentiation into an epithelial cell population, comprising further allowing an epithelial progenitor cell/stem cell population into which differentiation has been induced by the method as described above, to differentiate into an epithelial cell population.

In said method, examples of the epithelial cell population include a corneal epithelial cell population, an oral mucosal epithelial cell population, a urinary bladder epithelial cell population, a conjunctival epithelial cell population, a gastric mucosal epithelial cell population, a small intestinal epithelial cell population, a large intestinal epithelial cell population, a renal epithelial cell population, a renal tubular epithelial cell population, a gingival mucosal epithelial cell population, an esophagus epithelial cell population, a hepatic epithelial cell population, a pancreatic epithelial cell population, a pulmonary epithelial cell population, and a gallbladder epithelial cell population.

The method as described above may further comprise the step of isolating a keratin 14-positive and p63-positive cell population.

According to the fourth embodiment, the present invention provides a method for inducing differentiation into a keratin 12-positive corneal epithelial cell population from the epithelial progenitor cell/stem cell population, comprising continuing to culture the induced pluripotent stem cells in the method according to the first or second embodiment.

The method may further comprise the step of isolating a keratin 12-positive and keratin 14-negative cell population.

According to the fifth embodiment, the present invention provides cultures comprising an epithelial progenitor cell/stem cell population obtained by the method of the present invention and/or an epithelial cell population induced from said epithelial progenitor cell/stem cell population. A preferable form of the cultures is cultures comprising an epithelial progenitor cell/stem cell population and/or a corneal epithelial cell population obtained by the method of the present invention.

According to the sixth embodiment, the present invention provides a cell preparation for epithelial disease comprising an epithelial progenitor cell/stem cell population obtained by the method of the present invention and/or an epithelial cell population induced from said epithelial progenitor cell/stem cell population. A preferable form of the cell preparation is a cell preparation for epithelial disease comprising an epithelial progenitor cell/stem cell population and/or a corneal epithelial cell population obtained by the method of the present invention.

According to the sixth embodiment, the present invention provides a cell sheet comprising layers of an epithelial progenitor cell/stem cell population obtained by the method of the present invention and/or an epithelial cell population induced from said epithelial progenitor cell/stem cell population. A preferable form of the cell sheet is a cell sheet comprising layers of an epithelial progenitor cell/stem cell population and/or a corneal epithelial cell population obtained by the method of the present invention.

For the sheet of the present invention, it is preferred that the layers should be obtained by layered-culturing the cells.

### Advantageous Effects of Invention

Epithelial stem cells/progenitor cells or corneal epithelial-like cells of the present invention are derived from patient's own cells and thus are free from concerns about rejection. A layered corneal epithelial cell sheet prepared using the corneal epithelial-like cells of the present invention can be used as safe artificial cornea. Specifically, according to the present invention, the problems of donor shortage and rejection can be solved simultaneously in the field of regenerative medicine for corneal epithelial disease. Moreover, the cells of the present invention are not derived from ES cells but are obtained using, as a cell source, induced pluripotent stem cells prepared from patient's own somatic cells, and thus are free from ethical problems.

Not only corneal epithelial cells but also epidermal cells or various epithelial layers such as oral mucosal epithelium can be regenerated using the epithelial stem cells/progenitor cells of the present invention as a cell source. Specifically, the present invention is applicable as a basic technique for autologous regenerative medicine techniques for various epithelial diseases. Furthermore, an epithelial cell bank capable of reducing rejection can also be prepared by developing epithelial cells on a HLA genotype basis using this technique.

### BRIEF DESCRIPTION OF DRAWINGS

FIG. 1 shows the induction of the differentiation of mouse iPS cells into epithelial cells by a modified KCM method (7, 10, 17, and 27 days after induction).

FIG. 2 shows the induction of the differentiation of mouse iPS cells into corneal epithelial cells by the modified KCM method (17 days after induction) (*: keratin 12-positive corneal epithelial cells).

FIG. 3 shows BMP4 addition-induced increase in the epithelial induction efficiency of the modified KCM method (day 28) (FIGS. 3a to 3d: the induction of epithelial marker keratin 14-positive and p63-positive epithelial progenitor cells/stem cells by the addition of BMP4; and FIG. 3e: a result of flow cytometry analysis (increase in epithelial induction efficiency: 2.9%→6.0%)).

FIG. 4 shows results of inducing epithelial progenitor cells/stem cells or corneal epithelial cells by the modified KCM method using 3T3 cells as feeders (day 28).

FIG. 5 shows the induction of epithelial progenitor cells/stem cells or corneal epithelial cells by a modified SDIA method using PA6 cells as feeders (FIGS. 5a to 5c: results of 8-day culture in a differentiation medium; and FIGS. 5d to 5f: results of additional 3-day culture in an epithelial induction medium).

FIG. 6 shows the comparison of induction of epithelial progenitor cells/stem cells or corneal epithelial cells by the modified SDIA method between PA6 cells and 3T3 cells used as feeders (day 22) (FIGS. 6a to 6c: 3T3 cells; and FIGS. 6d to 6f: PA6 cells).

FIG. 7 shows the induction of epithelial progenitor cells/stem cells or corneal epithelial cells by the modified KCM method from human iPS cells (day 15) (FIG. 7a: keratin 14, FIG. 7b: keratin 3, and FIG. 7c: keratin 12).

FIG. 8 shows the induction of epithelial progenitor cells/stem cells by the modified SDIA method from human iPS cells (day 15) (FIG. 8a: PA6, FIG. 8b: 3T3, and FIG. 8c: 3T3).

FIG. 9 shows results of examining the influence of retinoic acid (RA) on the induction of the differentiation of mouse iPS cells and ES cells into epithelial cells by immunostaining. In the diagram, FIGS. 9A to 9C: mouse iPS (KCM medium), FIGS. 9D to 9F: mouse iPS (KCM medium supplemented with 0.5 nM BMP4), FIGS. 9G to 9I: mouse iPS (KCM medium supplemented with 0.5 nM BMP4+1 μM retinoic acid (RA)), FIGS. 9J to 9L: mouse ES (KCM medium supplemented with 0.5 nM BMP4+1 μM retinoic acid (RA)). Left boxes: p63, middle boxes: K14, and right boxes p63/K14.

FIG. 10 shows results of examining the influence of retinoic acid (RA) on the induction of the differentiation of mouse iPS cells into epithelial cells by real-time PCR. In each graph, FIG. 10A: Oct3/4, FIG. 10B: Nanog, FIG. 10C: ΔNp63, FIG. 10D: keratin 14 (K14). ▪: KCM medium, ▴: KCM medium supplemented with 0.5 nM BMP4, and ♦: KCM medium supplemented with 0.5 nM BMP4+1 μM retinoic acid (RA).

FIG. 11 shows results of examining the influence of retinoic acid on the induction of the differentiation of human iPS cells into epithelial cells by immunostaining (culture on 3T3 feeders).

FIG. 11A: differentiation medium+KCM medium, retinoic acid-supplemented, day 15. FIG. 11B: differentiation medium+KCM medium, retinoic acid-supplemented, day 29. FIG. 11C: control (differentiation medium+KCM medium, retinoic acid-free), day 15. FIG. 11D: differentiation medium+epithelial induction medium, retinoic acid-supplemented, day 15. In all the diagrams, upper boxes: K14, lower left boxes: p63, and lower right boxes: p63+K14.

FIG. 12 shows results of examining the influence of retinoic acid on the induction of the differentiation of human iPS cells into epithelial cells by immunostaining (culture on PA6 feeders, retinoic acid-supplemented, day 15; left box: phase-contrast microscope image, and lower right box: p63.

The specification of the present application encompasses the contents described in the specification of Japanese Patent Application No. 2009-120053 that serves as a basis for the priority of the present application.

### DESCRIPTION OF EMBODIMENTS

The present invention relates to: a method for inducing differentiation into an epithelial progenitor cell/stem cell population or a corneal epithelial cell population from induced pluripotent stem cells induced from mammalian somatic cells or undifferentiated stem cells; and a use of a cell population induced by said method in the treatment of disease in epithelial tissues.

### 1. Definitions

Hereinafter, some terms according to the present invention will be described.

### (1) Induced Pluripotent Stem Cell

The term “induced pluripotent stem cell” according to the present invention refers to a cell that has been reprogrammed (initialized) to have pluripotency similar to ES cells by introducing the defined factors to mammalian somatic cells or undifferentiated stem cells.

The “induced pluripotent stem cell” was established for the first time by Yamanaka et al. by introducing four factors (Oct3/4, Sox2, Klf4, and c-Myc) to mouse fibroblasts and designated as an “iPS cell (induced Pluripotent Stem Cell)” (Takahashi K, Yamanaka S., Cell, (2006) 126: 663-676). Thereafter, human iPS was also established by introducing these four factors to human fibroblasts (Takahashi K, Yamanaka S., et al. Cell, (2007) 131: 861-872.). Furthermore, a method for establishing more highly safe iPS cells that less induce oncogenesis was also successfully established, such as a c-Myc-free method (Nakagawa M, Yamanaka S., et al. Nature Biotechnology, (2008) 26, 101-106).

Thomson et al. from the University of Wisconsin have successfully established induced pluripotent stem cells prepared by introducing four genes of OCT3/4, SOX2, NANOG, and LIN28 to human fibroblasts (Yu J., Thomson JA. et al., Science (2007) 318: 1917-1920.). Moreover, Daley et al. from the Harvard University have reported the establishment of induced pluripotent stem cells prepared by introducing six genes of OCT3/4, SOX2, KLF4, C-MYC, hTERT, and SV40 large T to skin cells (Park I H, Daley G Q. et al., Nature (2007) 451: 141-146).

Sakurada et al. have reported induced pluripotent stem cells more efficiently induced by introducing Oct3/4, Sox2, Klf4, and c-Myc, and the like to, not somatic cells, but undifferentiated stem cells present in tissues after birth, as a cell source (Japanese Patent Laid-Open No. 2008-307007).

In addition, there are reports as to induced pluripotent stem cells prepared by introducing OCT3/4, KLF4, and low-molecular-weight compounds to mouse neural progenitor cells or the like (Shi Y., Ding S., et al., Cell Stem Cell, (2008) Vol. 3, Issue 5, 568-574), induced pluripotent stem cells prepared by introducing OCT3/4 and KLF4 to mouse neural stem cells endogenously expressing SOX2 and C-MYC (Kim J B., Scholer H R., et al., Nature, (2008) 454, 646-650), and induced pluripotent stem cells prepared using a DNMT inhibitor or HDAC inhibitor without the use of C-MYC (Huangfu D., Melton, D A., et al., Nature Biotechnology, (2008) 26, No. 7, 795-797).

Examples of known patents relating to induced pluripotent stem cells, including the patents as described above, can include Japanese Patent Laid-Open No. 2008-307007, Japanese Patent Laid-Open No. 2008-283972, US2008-2336610, US2009-047263, WO2007-069666, WO2008-118220, WO2008-124133, WO2008-151058, 2009-006930, WO2009-006997, and WO2009-007852.

The term “induced pluripotent stem cell” used in the present invention includes all of induced pluripotent stem cells known in the art and induced pluripotent stem cells equivalent thereto as long as these induced pluripotent stem cells satisfy the definition described at the onset and do not impair the object of the present invention. A cell source, introduced factors, an introduction method, and so on is not particularly limited.

Preferably, the cells are derived from a human and, more preferably, derived from a patient himself or herself in need of treatment using an epithelial or epidermal cell population including an epithelial progenitor cell/stem cell population or corneal epithelial cells induced from said cells.

### (2) Epithelial Progenitor Cell/Stem Cell

The term “epithelial progenitor cell/stem cell” according to the present invention means a population of undifferentiated epithelial cells that express no differentiation marker and are highly capable of proliferation. The “epithelial progenitor cell/stem cell” of the present invention is characterized by the expressions of a basal epithelial cell marker keratin 14 and an epithelial progenitor cell/stem cell marker p63.

### (3) Corneal Epithelial Cell

The cornea has a trilayer structure of a corneal epithelial layer, a corneal parenchymal layer, and a corneal endothelial layer from the surface. The term “corneal epithelial cell” according to the present invention is a cell that constitutes the outermost layer of this cornea and is composed of 4 or 5 corneal epithelial cell layers. The “corneal epithelial cell” is derived from the epidermal ectoderm. The parenchyma and endothelium of the cornea are derived from the neural crest and thought to contain stem cells separate from each other. The “corneal epithelial cell” according to the present invention is characterized by the expression of a corneal epithelial differentiation marker keratin 12.

### (4) Feeder Cell

The term “feeder cells (or also abbreviated to “feeders”)” used in the present invention means cells that are of a kind different from that of cultured cells and are used for assisting or adjusting culture conditions for cells of interest. Usually, the feeder cells are pretreated with γ-ray irradiation or an antibiotic such as mitomycin C (MMC) to prevent the feeder cells themselves from proliferating.

The feeder cells differ depending on the purpose of an experiment or the kind of the cells. For example, MEF (mouse embryonic fibroblast) or SNL (mouse embryo-derived fibroblast line) is used for ES cells or iPS cells.

Also in a differentiation induction method of the present invention, various feeder cells such as stromal cells and fibroblasts and coating with Matrigel, amnion, type I collagen, fibronectin, laminin, or the like can be used for a method modified from a KCM method described later.

On the other hand, stromal cells are used for a method modified from an SDIA method. 3T3 cells are preferable in terms of differentiation efficiency.

### (5) Stromal Cell and Stromal Cell-Derived Differentiation Factor

The term “stromal cells” used in the present invention means cells that support blood cells present in the bone marrow. The “stromal cells” proliferate while adhering to walls, unlike blood cells proliferating in a floating state by culture. The “stromal cells” are mesenchymal cells and are rich in stem cells which differentiate into various cells.

The “stromal cells” are rich in stem cells and are capable of pluripotent differentiation by themselves. Thus, their application to regenerative medicine has been expected. However, in the present invention, the “stromal cells” are used as feeder cells or the like for promoting the induction of differentiation into an epithelial progenitor cell/stem cell population or corneal epithelial cells from induced pluripotent stem cells.

The “stromal cells” are known to secrete a factor that controls cell differentiation. The term “stromal cell-derived differentiation factor” used in the present invention means such a factor that is secreted by stromal cells and controls cell differentiation. The “stromal cell-derived differentiation factor” has been confirmed to be able to selectively induce the differentiation of ES cells into nerve cells by culturing the ES cells with mouse bone marrow-derived stromal cells as described later, although the entity of the differentiation factor still remains to be elucidated. A method for inducing differentiation into nerve cells using such stromal cells or a stromal cell-derived differentiation factor was designated as an SDIA method (Kawasaki, H., Sasai, Y. et al., Neuron, (2000) 28, 31-40; Kawasaki, H., Sasai, Y. et al., Proc. Natl. Acad. Sci. USA, (2002) 99, 1580-1585; and Mizuseki, K., Sasai, Y. et al., Proc. Natl. Acad. Sci., USA, (2003) 100, 5828-5833).

(6) Cell Markers: Keratin 14, p63, and Keratin 12

In the present invention, a marker specific for each cell species is used for identifying cells into which differentiation has been induced. Specifically, the epithelial progenitor cells/stem cells according to the present invention are identified based on keratin 14-positive and p63-positive, while the corneal epithelial cells are identified based on keratin 12-positive and keratin 3-positive.

Keratin 14 (or cytokeratin 14: K14): The keratin 14 is a typical marker for basal epithelial cells.

p63: The p63, a cell nuclear protein belonging to the p53 gene family, is a typical marker for epithelial progenitor cells/stem cells. Its expression is observed in normal human epidermis and hair follicle basal cells and the like.

Keratin 12 (or cytokeratin 12: K12): The keratins 12 and 3 are typical differentiation markers for the corneal epithelium.

### 2. Differentiation Induction Method

In the present invention, differentiation into an epithelial progenitor cell/stem cell population or a corneal epithelial cell population is induced from induced pluripotent stem cells based on two methods described in detail below.

In this context, the induced pluripotent stem cells are cultured in advance using an appropriate medium (commercially available medium for ES cells, medium for iPS cells, etc.) on feeder cells such as MEF or SNL.

### 2.1 Modification of KCM Method

KCM (Keratinocyte Culture Medium) is an abbreviation of a medium for epidermal keratinocyte culture. A KCM medium, a KSFM medium (Invitrogen), Epi-life (Cascade Biologics), a 3T3-conditioned medium, and the like are known as media for epidermal cells. The KCM medium is discriminated from other media for epidermal keratinocytes in terms of cholera toxin, fetal bovine serum, hydrocortisone, and usual calcium concentration. In the present specification, a method for inducing differentiation into epidermal cells using this KCM medium is referred to as a KCM method.

The present inventors successfully induced differentiation into an epithelial progenitor cell/stem cell population from induced pluripotent stem cells by applying a modification of this KCM medium. In this context, the epidermal keratinocytes are limited to epithelial cells in the skin. In general, epidermal cells have, for example, the properties of being keratinized and expressing markers such as keratin 1 and keratin 10 and are one kind of differentiated form among epithelial cells. Therefore, epidermal corneal cells are not identical to epithelial cells.

In general, in a culture method using the KCM medium, epidermal keratinocytes are cultured with collagen as a support. However, the present inventors have confirmed that the more favorable induction of differentiation into an epithelial progenitor cell/stem cell population and corneal epithelial cells can be achieved by using feeder cells.

Specifically, the induced pluripotent stem cells are cultured on feeder cells or a support selected from collagen, basement membrane matrix (Matrigel (registered trademark)), amnion, fibronectin, and laminin using a medium for epidermal cells containing an epidermal growth factor, cholera toxin, and serum (e.g., fetal bovine serum) and thereby induced to differentiate into a keratin 14-positive and p63-positive epithelial progenitor cell/stem cell population. It is preferred that the medium further contains hydrocortisone, insulin, transferrin, selenium, and so on. Moreover, the collagen is preferably type I collagen or type IV collagen. Atelocollagen free from antigenicity is preferable.

The feeder cells used are not particularly limited. For example, stromal cells or fibroblasts can be used. Particularly, the stromal cells are preferable. A preferable example thereof can include 3T3 cells.

The 3T3 cells are a cell line of cultured fibroblasts derived from mouse skin. The name is derived from “3 days, transfer, inoculum 3×10⁵ cells/50 mm dish”, i.e., the property of maintaining their functions by inoculating a relatively large number of cells and subculturing the cells in a short culture period.

The 3T3 cells include some cell lines such as Swiss/3T3, 3T3-swiss albino, BALB/3T3, and NIH/3T3. Any of these cell lines may be used.

Any medium that can be used in animal cell culture, such as DMEM, BME, α-MEM, Dulbecco MEM, BGJb, CMRL 1066, Glasgow MEM, Improved MEM Zinc Option, IMDM, Medium 199, Eagle MEM, Ham's, RPMI 1640, Fischer's, McCoy's, and William's E media and a mixed medium thereof, can be used as a basic medium for the KCM medium used in the method. The KCM medium is prepared by adding, to this basic medium, various nutrients necessary for cell maintenance and growth and each component necessary for differentiation induction.

Examples of the nutrients can include carbon sources (e.g., glycerol, glucose, fructose, sucrose, lactose, honey, starch, and dextrin), fatty acid, oil and fat, lecithin, hydrocarbons (e.g., alcohols), nitrogen sources (e.g., ammonium sulfate, ammonium nitrate, ammonium chloride, urea, and sodium nitrate), inorganic salts (e.g., common salt, potassium salt, phosphate, magnesium salt, calcium salt, iron salt, and manganese salt), monopotassium phosphate, dipotassium phosphate, magnesium sulfate, sodium chloride, ferrous sulfate, sodium molybdate, sodium tungstate, manganese sulfate, various vitamins, and amino acids.

Examples of the component that promotes differentiation induction can include antibiotics such as penicillin and streptomycin, cholera toxin, transferrin, insulin, EGM (Epidermal Growth Factor), serum or a serum substitute, and KSR (Knockout Serum Replacement).

The pH of the medium obtained by formulating these components is in the range of 5.5 to 9.0, preferably 6.0 to 8.0, more preferably 6.5 to 7.5.

The culture is performed under conditions involving 36° C. to 38° C., preferably 36.5° C. to 37.5° C., 1% to 25% 0₂, and 1% to 15% CO₂.

More favorable induction of differentiation into an epithelial progenitor cell/stem cell population can be achieved by adding BMP4 (Bone Morphogenetic Protein 4) to the medium. BMP4, one of bone morphogenetic factors, belongs to the transforming growth factor-β (TGF-β) superfamily. This BMP4 is known to modulate differentiation, growth, and various cell functions and known to suppress differentiation into nerves and promote differentiation into epidermal cells.

Further favorable induction of differentiation into an epithelial progenitor cell/stem cell population can be achieved by further adding retinoic acid to the medium. The retinoic acid, one kind of vitamin A derivative, is known to participate in the control of differentiation/growth of various cells, such as the promotion of differentiation/growth of epidermal cells. In this context, the retinoic acid may be a salt or derivative thereof usually used.

The induced pluripotent stem cells may be cultured in an aggregated state to form an embryoid body. In terms of differentiation efficiency, it is preferred that the induced pluripotent stem cells should be induced to differentiate without aggregation or embryoid body formation.

### 2.2 Modification of SDIA (Stromal Cell-Derived Inducing Activity) Method

The SDIA method is an abbreviation of stromal cell-derived inducing activity method as described above and is known to induce nerve cells from ES cells using a differentiation factor secreted by stromal cells (supra).

The present inventors have successfully induced differentiation into an epithelial progenitor cell/stem cell population from induced pluripotent stem cells by applying a modification of this SDIA method. Although both epithelial cells and nerve cells are cells derived from the ectoderm, the nerve is derived from the neural ectoderm while the epithelial cells are derived from the epidermal ectoderm. Moreover, these cells are totally different cell lineages in terms of functions and morphology.

Usually, a stromal cell line called PA6 is used in the SDIA method. However, the present inventors compared PA6 and 3T3 cells and consequently confirmed that the induction efficiency of differentiation into epithelial stem cells/progenitor cells was significantly improved by using 3T3 cells as feeders. Moreover, the induction efficiency of differentiation into epithelial stem cells/progenitor cells was higher in the presence of serum. When PA6 cells were used as feeders, the induced pluripotent stem cells could be induced to differentiate into epithelial stem cells/progenitor cells, as in 3T3 feeders, by adding a promoter such as retinoic acid.

In the present invention, the induced pluripotent stem cells are cultured on 3T3 cells or in the presence of a 3T3 cell-derived differentiation factor and thereby induced to differentiate into a keratin 14-positive and p63-positive epithelial progenitor cell/stem cell population.

Any medium that can be used in animal cell culture, such as DMEM, BME, BGJb, CMRL 1066, Glasgow MEM, Improved MEM Zinc Option, IMDM, Medium 199, Eagle MEM, α-MEM, Dulbecco MEM, Ham's , RPMI 1640, Fischer's, McCoy's, William's E media and a mixed medium thereof, can be used as a basic medium for the medium used. The medium is prepared by adding, to this basic medium, various nutrients necessary for cell maintenance and growth, and each component necessary for differentiation induction.

Examples of the nutrients can include carbon sources (e.g., glycerol, glucose, fructose, sucrose, lactose, honey, starch, and dextrin), fatty acid, oil and fat, lecithin, hydrocarbons (e.g., alcohols), nitrogen sources (e.g., ammonium sulfate, ammonium nitrate, ammonium chloride, urea, and sodium nitrate), inorganic salts (e.g., common salt, potassium salt, phosphate, magnesium salt, calcium salt, iron salt, and manganese salt), monopotassium phosphate, dipotassium phosphate, magnesium sulfate, sodium chloride, ferrous sulfate, sodium molybdate, sodium tungstate, manganese sulfate, various vitamins, and amino acids.

In addition, examples of optional components can include pyruvic acid, pyruvic acid, amino acid reducing agents (e.g., β-mercaptoethanol), and serum or a serum substitute. In this context, examples of the serum substitute include albumin (e.g., lipid-rich albumin), transferrin, fatty acid, insulin, collagen precursors, trace elements, β-mercaptoethanol or 3′-thiolglycerol, commercially available Knockout Serum Replacement (KSR), Chemically-defined Lipid concentrated (manufactured by Gibco), and Glutamax (manufactured by Gibco).

The pH of the medium obtained by formulating these components is in the range of 5.5 to 9.0, preferably 6.0 to 8.0, more preferably 6.5 to 7.5.

The culture is performed under conditions involving 36° C. to 38° C., preferably 36.5° C. to 37.5° C., 1% to 25% O₂, and 1% to 15% CO₂.

In terms of differentiation efficiency, it is preferred that the induced pluripotent stem cells should be cultured in a differentiation medium containing a serum substitute and/or BMP4 and then cultured in an epithelial induction medium containing serum such as fetal bovine serum and/or BMP4 or a medium for epidermal cells (e.g., a KCM medium) containing an epidermal growth factor and/or cholera toxin and serum. It is preferred that the differentiation medium and the epithelial induction medium or the medium for epidermal cells should further contain nonessential amino acid, β-mercaptoethanol, sodium pyruvate, and the like. In this context, examples of the serum substitute include albumin (e.g., lipid-rich albumin), transferrin, fatty acid, insulin, collagen precursors, trace elements, β-mercaptoethanol or 3′-thiolglycerol, commercially available Knockout Serum Replacement (KSR), Chemically-defined Lipid concentrated (manufactured by Gibco), and Glutamax (manufactured by Gibco). Moreover, the nonessential amino acid means an amino acid other than essential amino acids (amino acids that cannot be synthesized in vivo by the animals and must be ingested as nutrients). For humans, 11 amino acids, i.e., asparagine, aspartic acid, arginine, glutamine, glutamic acid, glycine, proline, ornithine, tyrosine, serine, and alanine, correspond to nonessential amino acids. In the present invention, the “nonessential amino acid” does not have to include all of these 11 amino acids and may be some of them. Preferably, 5 or more amino acids that are not contained in the basic medium, including asparagine, aspartic acid, proline, ornithine, and alanine, can be contained therein.

Further favorable induction of differentiation into an epithelial progenitor cell/stem cell population can be achieved by adding retinoic acid to the epithelial induction medium or the medium for epidermal cells. Retinoic acid can be added to not only the epithelial induction medium but also the differentiation medium. As described above, the retinoic acid may be a salt or derivative thereof usually used.

Any of these media are based on the component composition as described above. The differentiation medium is free from fetal bovine serum and is considered to mainly contribute to the growth of undifferentiated cells. The epithelial induction medium is a medium containing fetal bovine serum and is characterized, for example, by promoting differentiation into epithelial cells. A specific example of the medium for epidermal cells is a KCM medium.

3. Induction of Differentiation from Epithelial Progenitor Cell/Stem Cell Population
3.1 Induction of Differentiation into Epithelial Cells

The epithelial progenitor cell/stem cell population into which differentiation has been induced by the method of the present invention can be allowed to differentiate into various other epithelial cell populations.

Examples of the epithelial cell populations into which the epithelial progenitor cell/stem cell population can be induced to differentiate can include a corneal epithelial cell population, an epidermal cell population, a hair follicle cell population, an oral mucosal epithelial cell population, a urinary bladder epithelial cell population, a conjunctival epithelial cell population, a gastric mucosal epithelial cell population, a small intestinal epithelial cell population, a large intestinal epithelial cell population, a renal epithelial cell population, a renal tubular epithelial cell population, a gingival mucosal epithelial cell population, an esophagus epithelial cell population, a hepatic epithelial cell population, a pancreatic epithelial cell population, a pulmonary epithelial cell population, and a gallbladder epithelial cell population.

3.2 Induction of Differentiation into Corneal Epithelial Cells

Any of the two methods of the present invention (modified KCM method and modified SDIA method) can induce differentiation into a keratin 12-positive and keratin 14-negative corneal epithelial cell population from the epithelial progenitor cell/stem cell population by continuing to culture for a fixed period. For example, differentiation from iPS cells into corneal epithelial cells can be induced by a method for inducing differentiation into corneal epithelial cells from epidermal cells, comprising coculturing said cell population with limbal fibroblasts (Blazejewska E A et al., Stem Cells, (2009) Mar; 27 (3): 642-652).

The culture period for inducing differentiation into a corneal epithelial cell population is appropriately determined depending on the kind of the cells used and culture conditions.

### 4. Cell Isolation (Purification)

### 4.1 Isolation of Epithelial Progenitor Cell/Stem Cell Population

The epithelial progenitor cell/stem cell population into which differentiation has been induced by the method of the present invention can be isolated using its markers keratin 14 and p63.

This isolation can be easily carried out using an antibody specific for each marker according to a routine method. For example, the cell population may be isolated by separation using antibody-labeled magnetic beads, antibody-immobilized columns, or a cell sorter (FACS) using a fluorescently labeled antibody. The antibody used may be a commercially available one or may be prepared according to a routine method.

Specifically, anti-integrin α₆ antibody- and anti-E-cadherin antibody-immobilized immunomagnetic beads are respectively prepared, and a fraction binding to both of the beads is separated. Alternatively, the cell population can be separated by column chromatography using anti-integrin α₆ antibody- and anti-E-cadherin antibody-immobilized carriers, or integrin α₆-positive and E-cadherin-positive cells can also be separated by FACS.

### 4.2 Isolation of Corneal Epithelial Cell Population

The corneal epithelial cell population into which differentiation has been induced by the method of the present invention can also be isolated using a method for culturing the corneal epithelial cells.

Specifically, the corneal epithelial cell population into which differentiation has been induced as described above is collected by trypsin treatment. The collected cells can be inoculated again into a medium for epithelial cell culture such as a KCM or KSFM (Invitrogen) medium (3T3 cells are used as feeders for the KCM medium), then cultured, and further repetitively subcultured to thereby purify corneal epithelial cells.

### 5. Use in Regenerative Medicine

### 5.1 Cultures

Cultures containing an epithelial progenitor cell/stem cell population and/or an epidermal or epithelial cell population obtained by the method of the present invention can be used in research or regenerative medicine or as a raw material for a cell preparation described later.

### 5.2 Cell Preparation for Treatment of Epithelial Disease

The epithelial progenitor cell/stem cell population isolated after differentiation induction by the method of the present invention and/or the epidermal or epithelial cell population can be used as a cell preparation for epithelial disease.

A method for administering the cell preparation of the present invention is not particularly limited and is possibly local transplantation by surgical means, intravenous administration, administration by lumbar puncture, local injection, subcutaneous administration, intradermal administration, intraperitoneal administration, intramuscular administration, intracerebral administration, intracerebroventricular administration, or intravenous administration, or the like, according to a site to which the cell preparation is applied.

The cell preparation of the present invention may contain any components for cell maintenance/growth, scaffoldings or ingredients for assisting administration to an affected part, and other pharmaceutically acceptable carriers.

Examples of the ingredients necessary for cell maintenance/growth include: medium components such as carbon sources, nitrogen sources, vitamins, minerals, salts, and various cytokines; and extracellular matrix preparations such as Matrigel™.

Examples of the scaffoldings or ingredients for assisting administration to an affected part include: biodegradable polymers such as collagen, polylactic acid, hyaluronic acid, cellulose, and derivatives thereof, and a complex of two or more of them; and injectable aqueous solutions such as saline, media, physiological buffers (e.g., PBS), and isotonic solutions (e.g., D-sorbitol, D-mannose, D-mannitol, and sodium chloride) containing glucose or other auxiliaries. These scaffoldings or ingredients may be used in combination with an appropriate solubilizer, for example, alcohol (specifically, ethanol) or polyalcohol (e.g., propylene glycol or polyethylene glycol), a nonionic surfactant, for example, polysorbate 80 or HCO-50.

In addition, the cell preparation may optionally contain pharmaceutically acceptable organic solvents, polyvinyl alcohol, polyvinylpyrrolidone, carboxyvinyl polymers, sodium carboxymethylcellulose, sodium polyacrylate, sodium alginate, water-soluble dextran, sodium carboxymethyl starch, pectin, methylcellulose, ethylcellulose, xanthan gum, gum arabic, casein, agar, polyethylene glycol, diglycerin, glycerin, propylene glycol, Vaseline, paraffin, stearyl alcohol, stearic acid, mannitol, sorbitol, lactose, and a surfactant, a buffer, an emulsifier, a suspending agent, a soothing agent, a stabilizer, and the like acceptable as pharmaceutical additives.

Actual additives are selected from among the additives alone or in appropriate combination according to the dosage form of the therapeutic agent of the present invention, but are not limited to them. For example, when the cell preparation of the present invention is used as an injectable preparation, the purified antibody is dissolved in a solvent, for example, saline, a buffer, or a glucose solution, and this solution can be supplemented with an anti-adsorption agent, for example, Tween 80, Tween 20, or gelatin and then used.

Examples of the disease that may be targeted by the cell preparation of the present invention include Stevens-Johnson syndrome, ocular pemphigoid, thermal/chemical trauma, aniridia, Salzmann corneal degeneration, idiopathic corneal endotheliopathy, scars after trachoma, corneal trepanation, ulcer in the peripheral part of the cornea, corneal epithelial detachment after excimer laser treatment, narrowing after esophagus cancer treatment, and other keratoconjunctive, skin, oral mucosal, esophagus mucosal, or gastric mucosal diseases.

### 5.3 Layered Cell Sheet

The epithelial progenitor cell/stem cell population and/or the epithelial cell population, obtained by the method of the present invention, can be layered and prepared into a cultured epithelial cell sheet.

The layering of the cells can be carried out according to the previous reports of the present inventors (WO2004/069295, Japanese Patent Laid-Open No. 2005-130838, Nishida K et al., N. Engl. J. Med. (2004) 351: 1187-96, etc.). For example, the epithelial cell population into which differentiation has been induced by the method of the present invention using 3T3 cells or the other stromal cells as feeder cells is cultured in a medium for epithelial cell layering (e.g., a KCM medium). In this way, the epithelial cells can be layered-cultured to prepare a cultured epithelial cell sheet (Nishida K et al., N. Engl. J. Med. (2004) 351: 1187-96). Alternatively, the epithelial cell population into which differentiation has been induced by the method of the present invention is cultured on a porous membrane, and the epithelial cells can be layered such that a medium is constantly supplied via the porous membrane from a lower layer to prepare a cultured epithelial cell sheet (Japanese Patent Laid-Open No. 2005-130838).

### 6. Other Aspects

An epithelial cell bank capable of reducing rejection can also be prepared by developing epithelial cells on a HLA genotype basis using the method of the present invention. A technique for regenerative medicine by allotransplantation using such a cell bank is a field desired to be industrialized.

### Examples

Hereinafter, the present invention will be described specifically with reference to Examples, but not limited to these Examples.

### Example 1

### Induction of Differentiation into Epithelial Cells from Mouse iPS Cells

### 1. Culture of Mouse iPS Cells:

Mouse iPS cells were kindly provided by Professor S. Yamanaka from the Kyoto University (Okita K et al., Nature (2007) 448: 313-317). SNL (SNL76/7) was kindly provided by Dr. Allan Bradley from the Bayer College of Medicine. The mouse iPS cells were maintained with this SNL (SNL76/7) as feeders using a medium for SNL feeders shown below.

SNL cells treated with mitomycin (MMC) were inoculated to a gelatin-coated culture dish and used as feeder cells. The mouse iPS cells were inoculated thereonto and maintained at 37° C. in a 5% CO₂ atmosphere using a medium for iPS cell culture.

SNL feeder medium

DMEM (Nacalai Tesque)
7% FBS (Daiichi Chemical)
2 mM L-Glutamine (Invitrogen)
1% Penicillin-Streptomycin (Invitrogen, 100x)

Medium for iPS cell culture

DMEM (Nacalai Tesque)
15% FBS (Daiichi Chemical)
2 mM L-Glutamine (100x, Invitrogen)
1% Penicillin-Streptomycin (Invitrogen, 100x)
1 μg/ml Puromycin (Invitrogen)
1% nonessential amino acids (Invitrogen, 100x)
0.1% 2-mercaptoethanol (Invitrogen)

### 2. Preparation of Differentiation Induction System

### 2.1. KCM (Keratinocyte Culture Medium) Method

### (1) Culture on Collagen

The iPS cells on the SNL feeders were collected by treatment with 0.25% trypsin/EDTA and further pipetted to prepare an iPS cell suspension (single cell suspension). The obtained cell suspension was incubated on a gelatin-coated culture dish for approximately 1 to 2 hours. The supernatant was collected to thereby allow only the feeder cells to adhere to the dish and collect only the iPS cells. The number of the obtained iPS cells was counted. The iPS cells were inoculated at a density of 0.5 to 10×10³ cells/cm² onto a type IV collagen-coated culture dish as described below and cultured at 37° C. for 7 to 28 days in a 5% CO₂ atmosphere using a KCM medium shown below. 0.5 nM BMP4, (R&D System) was further added to the KCM medium, and the cells were cultured in the same way as above.

### <Coating Method>

Type IV collagen (Nitta Gelatin Inc.) was diluted 10-fold with dilute hydrochloric acid (pH 3). The diluted solution was spread as a thin layer over a culture dish and placed for 30 minutes or longer in a clean bench for drying. Before use, the culture dish was washed three times with phosphate-buffered saline (PBS) (Invitrogen).

Composition of KCM medium

69% Dulbecco's Modified Eagle's Medium (DMEM) (Sigma-Aldrich)
23% Nutrient Mixture F-12 Ham (Sigma-Aldrich)
5% Fetal Bovine Serum (FBS) (Japan Bio Serum)
1% Penicillin-Streptomycin (Invitrogen, 100x)
0.4 μg/ml Hydrocortisone Succinate (Wako)
2 nM 3,3′,5-Triiodo-L-thyronine sodium salt (MP BIOMEDICALS)
100 nM Cholera Toxin (Calbiochem)
2 mM L-Glutamine (Invitrogen)
0.5% Insulin Transferrin Selenium (GIBCO, 200x)
10 ng/ml Recombinant Human EGF (R&D Systems)

### (2) Culture on 3T3 Cells

iPS cells prepared in the same way as in the preceding paragraph were inoculated at a density of 0.1 to 10×10³ cells/cm² onto a culture dish inoculated with 3T3 cells treated with MMC as feeder cells, and cultured at 37° C. for 7 to 27 days. The cells were appropriately fixed with PFA. 0.5 nM BMP4 (R&D System) was further added to the KCM medium, and the cells were cultured in the same way as above.

### 2.2. SDIA (Stromal Cell-Derived Activity) Method

### (1) Culture on PA6 Cells

The iPS cells on the SNL feeders were collected by treatment with 0.25% trypsin/EDTA and further pipetted to prepare an iPS cell suspension (single cell suspension). The obtained cell suspension was incubated on a gelatin-coated culture dish for approximately 1 to 2 hours. The supernatant was collected to thereby allow only the feeder cells to adhere to the dish and collect only the iPS cells. The number of the obtained iPS cells was counted. The iPS cells were inoculated at a density of 0.1 to 10×10³ cells/cm² onto a culture dish inoculated with PA6 cells. The iPS cells were cultured at 37° C. for 8 days in a differentiation medium shown below in a 5% CO₂ atmosphere and subsequently cultured at 37° C. for 2 to 27 days in an epithelial induction medium. The cells were apprppriately fixed with PFA. Furthermore, difference was also evaluated between the presence and absence of addition of FBS to the epithelial induction medium.

Differentiation medium (modified SDIA method)

G-MEM (Invitrogen)
10% KSR (Invitrogen)
2 mM L-Glutamine (Invitrogen)
1% Pyruvate (Invitrogen, 100x)
1% nonessential amino acids (Invitrogen, 100x)
0.1% 2-mercaptoethanol (Invitrogen)
0.5 nM BMP-4 (R&D System)

### Epithelial Induction Medium (Modified SDIA Method)

Differentiation medium (−10% KSR) +10% FBS (Japan bio serum)* *A differentiation medium except for KSR was supplemented with 10% FBS and used as an epithelial induction medium.

(2) Culture on 3T3 cells

iPS cells prepared in the same way as in the preceding paragraph were inoculated at a density of 0.1 to 10×10³ cells/cm² onto a culture dish inoculated with 3T3 cells treated with MMC as feeder cells. The iPS cells were cultured at 37° C. for 8 days in a differentiation medium in a 5% CO₂ atmosphere and subsequently cultured at 37° C. for 2 to 27 days in an epithelial induction medium. The cells were appropriately fixed with PFA. Furthermore, difference was also evaluated between the presence and absence of addition of FBS to the epithelial induction medium.

3. Verification Of Cells into Which Differentiation had Been Induced

The cells after differentiation induction were examined for the expressions of a basal epithelial cell marker keratin 14, an epithelial progenitor cell/stem cell marker p63, and a corneal epithelial differentiation marker keratin 12 by an immunostaining method. Moreover, keratin 14-positive cells were analyzed by flow cytometry. The immunostaining method and the flow cytometry analysis will be shown in detail below.

### <Immunostaining Method>

Cytokeratin 14 (keratin 14 (K14))

After fixation in cold methanol (−30° C./20 min), 5% NST was added to the cells, which were then left at room temperature for 30 minutes for blocking. Then, the cells were reacted with a primary antibody (Cytokeratin 14 (AF64): Covance) overnight at 4° C., then washed with PBS, and reacted with a secondary antibody at room temperature for 2 hours. The cell nuclei were stained with Hoechst 33342.

Cytokeratin 12 (keratin 12 (K12))

After fixation in cold methanol (−30° C./20 min), 5% NST was added to the cells, which were then left at room temperature for 30 minutes for blocking. Then, the cells were reacted with a primary antibody (Cytokeratin 12 (L-15): Santa Cruz Biotechnology) overnight at 4° C., then washed with PBS, and reacted with a secondary antibody at room temperature for 2 hours. The cell nuclei were stained with Hoechst 33342.

Cytokeratin 3 (keratin 3 (K3))

After fixation in cold methanol (−30° C./20 min), 5% NST was added to the cells, which were then left at room temperature for 30 minutes for blocking. Then, the cells were reacted with a primary antibody (Cytokeratin 3/2p (AE5): R&D system) overnight at 4° C., then washed with PBS, and reacted with a secondary antibody at room temperature for 2 hours. The cell nuclei were stained with Hoechst 33342.

p63

After fixation in cold methanol (−30° C./20 min), 5% NST was added to the cells, which were then left at room temperature for 30 minutes for blocking. Then, the cells were reacted with a primary antibody (p63 (S-16): Santa Cruz Biotechnology) at 4° C. for 72 hours, then washed with PBS, and reacted with a secondary antibody at room temperature for 2 hours. The cell nuclei were stained with Hoechst 33342.

### <Flow Cytometry Analysis>

Cytokeratin 14

The cells were collected using 0.25% trypsin/EDTA. The collected cells were subjected to fixation and membrane permeabilization using Cytofix/Cytoperm kit (BD Biosciences). After the treatment, a primary antibody (Cytokeratin 14 (AF64): Covance) was diluted 1000-fold and added to the cells, which were then left standing at room temperature for 2 hours. Pellets were washed by centrifugation, and a secondary antibody (anti-rabbit Alexa 488) was diluted 200-fold and further added to the pellets, which were then left standing at room temperature for 1 hour. Pellets were washed by centrifugation and then suspended in 1 to 2 ml of PBS. The suspension was applied to a flow cytometer to examine the rate of keratin 14-positive cells.

### 3.1. Modified KCM Method

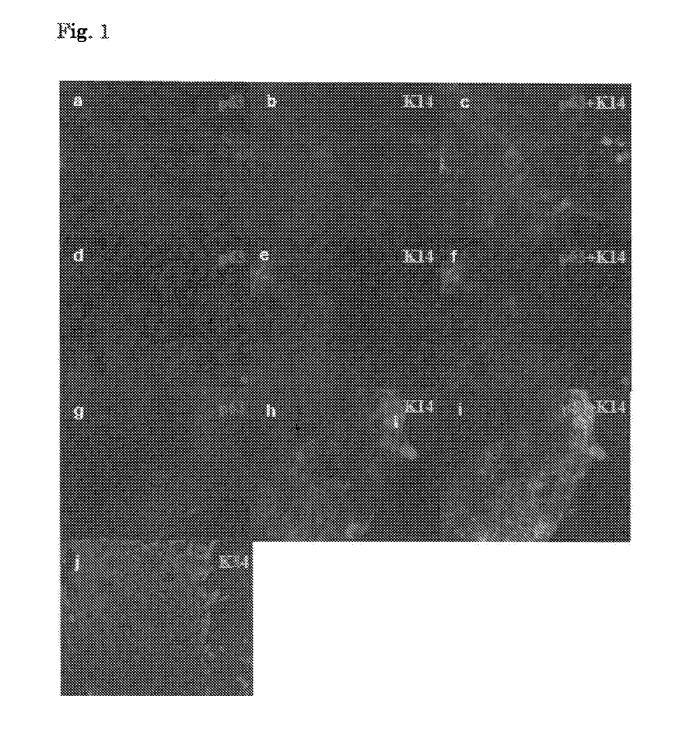

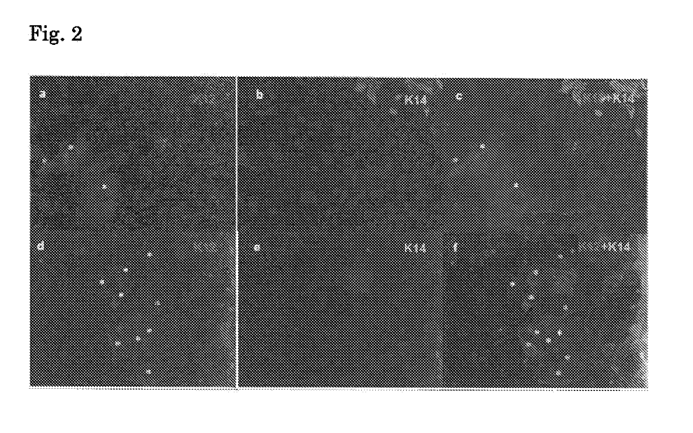

As a result of differentiation induction on collagen using the KCM medium, cells expressing the basal epithelial cell marker keratin 14 were observed from day 10 onward, and cells expressing both keratin 14 and the epithelial progenitor cell/stem cell marker p63 were observed from day 17 onward (FIG. 1). Moreover, keratin 14-negative epithelial cells expressing the corneal epithelial differentiation marker keratin 12 were observed from day 17 onward (FIGS. 2: corneal epithelial cells expressing corneal epithelial differentiation marker keratin 12 (FIGS. 2a and 2d), but not expressing keratin 14 (FIGS. 2b and 2e) were observed (FIGS. 2c and 2f)).

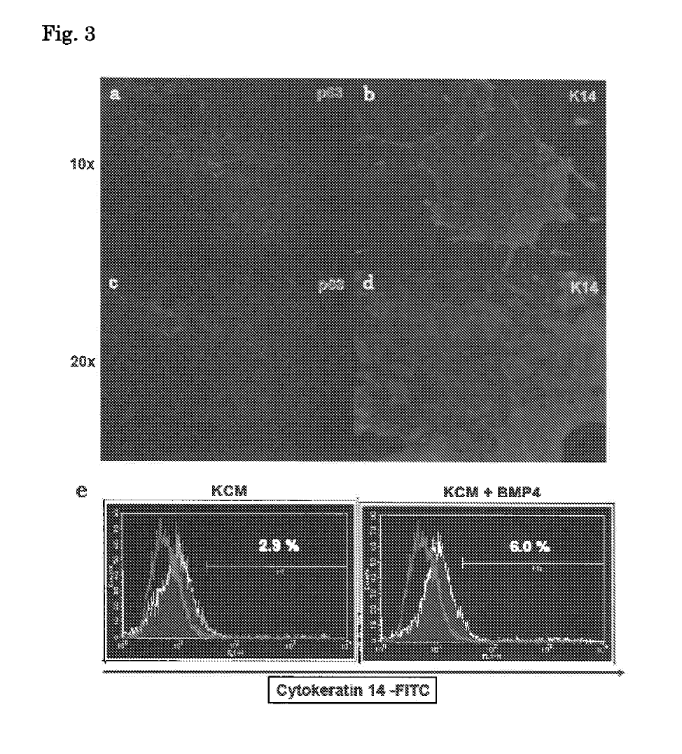

The addition of BMP4 to the culture system significantly increased epithelial induction efficiency (FIGS. 3a to 3d: induction into epithelial marker keratin 14-positive and p63-positive epithelial progenitor cells/stem cells by the addition of BMP4 (day 28)). As a result of flow cytometry analysis of keratin 14-positive cells, it was confirmed that the addition of BMP4 increased the induction efficiency of differentiation from 2.9% to 6.0% (FIG. 3e).

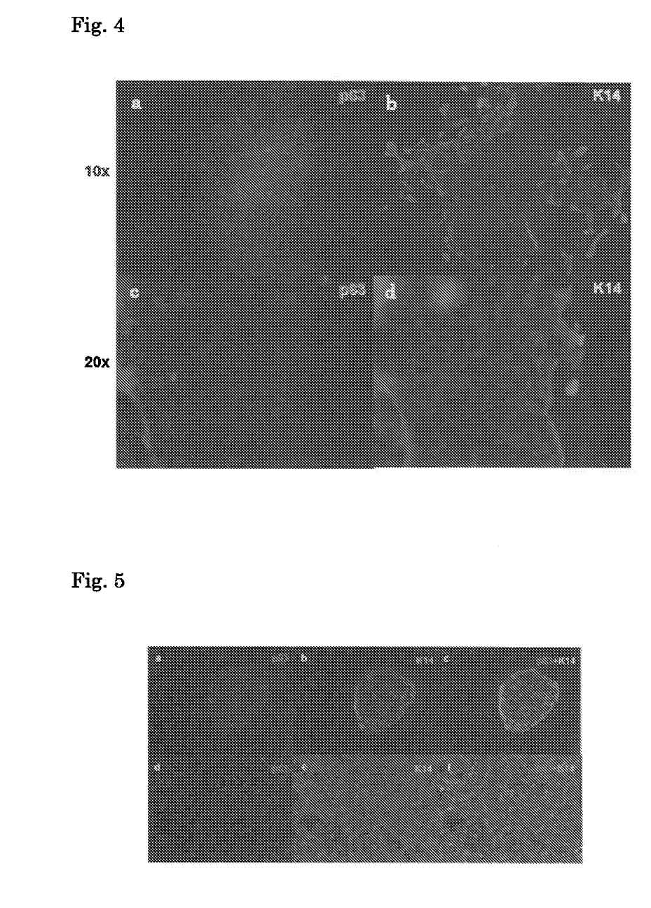

It was further confirmed that use of 3T3 cells as feeders instead of collagen improved the induction efficiency of differentiation (FIG. 4).

### 3.2. Modified SDIA Method

The cells were cultured for 8 days in a differentiation medium using PA6 cells as feeders (FIGS. 5a to 5c) and further cultured for 2 to 27 days in an epithelial induction medium (FIGS. 5d to 5f show results obtained at day 3). As a result, a plurality of epithelial cell colonies coexpressing p63 (FIGS. 5a and 5d) and keratin 14 (FIGS. 5b and 5e) were observed (FIG. 5c). It was further confirmed that the FBS-containing epithelial induction medium promoted differentiation into epithelial cells (FIGS. 5d to 5f).

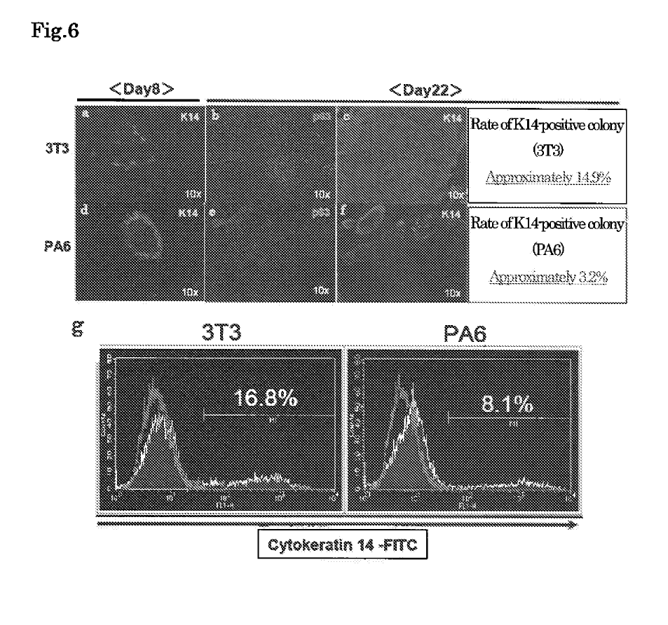

When 3T3 cells were used as feeders (FIGS. 6a to 6c), keratin 14-positive and p63-positive epithelial progenitor cell/stem cell colonies were efficiently induced compared with PA6 cells used as feeders (FIGS. 6d to 6f) (14.9% vs. 3.2%). As a result of flow cytometry analysis of keratin 14-positive cells, the induction efficiency of differentiation into epithelial progenitor cells/stem cells at day 22 was 16.8% and 8.1% for 3T3 cells and PA6 cells, respectively (FIG. 6g).

### 4. Discussion

These results demonstrated that mouse iPS cells can be induced to differentiate into epithelial stem cells/progenitor cells and corneal epithelial cells by the modified KCM method or the modified SDIA method. It was demonstrated that use of 3T3 cells as feeders significantly improved the induction efficiency of differentiation into epithelial stem cells/progenitor cells in both of the modified KCM method and the modified SDIA method.

It was also demonstrated that the addition of BMP4 in the modified KCM method or the addition of FBS to the epithelial induction medium in the modified SDIA method improved the induction efficiency of differentiation into epithelial stem cells/progenitor cells.

These results demonstrated that differentiation into epithelial stem cells/progenitor cells can be induced by the modified SDIA method.

### Example 2

### Induction of Differentiation into Epithelial Cells from Human iPS Cells

1. Culture of Human iPS cells:

Human iPS cells were kindly provided by Professor S. Yamanaka from the Kyoto University (Takahashi K, Yamanaka S., et al. Cell, (2007) 131: 861-872). The human iPS cells were maintained with MEF cells (KITAYAMA LABES CO., LTD.) as feeders using a medium for MEF feeders shown below.

Specifically, MEF cells treated with mitomycin were inoculated to a gelatin-coated culture dish and used as feeder cells. The human iPS cells were inoculated thereonto and maintained at 37° C. in a 5% CO₂ atmosphere using a medium for primate ES cells (Reprocell Inc.) supplemented with 4 ng/ml bFGF.

MEF feeder medium

DMEM (Nacalai Tesque)
10% FBS (Daiichi Chemical)
1% Penicillin-Streptomycin (Invitrogen, 100x)

### 2.1. Modified KCM (Keratinocyte Culture Medium) Method

### (1) Culture on Collagen

The human iPS cells on the MEF feeders were treated with 0.25% trypsin/EDTA to disrupt the iPS cell colonies. The cells were pipetted a few times to collect a cluster population of iPS cell colonies (not used as single cells). The obtained iPS cell colonies were incubated on a gelatin-coated culture dish in a KCM medium for approximately 1 to 2 hours. The supernatant was collected to thereby allow only the MEF feeder cells to adhere to the dish and collect only the human iPS cells. The number of the obtained human iPS cell colonies was counted. The iPS cells were inoculated at a density of 10 to 1000 colonies/cm² onto a type IV collagen-coated culture dish and cultured in a KCM medium (shown below) supplemented with 0.5 nM BMP4 (R&D System).

### <Coating Method>

Type IV collagen (Nitta Gelatin Inc.) was diluted 10-fold with dilute hydrochloric acid (pH 3). The diluted solution was spread as a thin layer over a culture dish and placed for 30 minutes or longer in a clean bench for drying. Before use, the culture dish was washed three times with phosphate-buffered saline (PBS) (Invitrogen).

Composition of KCM medium

69% Dulbecco's Modified Eagle's Medium (DMEM) (Sigma-Aldrich)
23% Nutrient Mixture F-12 Ham (Sigma-Aldrich)
5% Fetus Bovine Serum (FBS) (Japan Bio Serum)
1% Penicillin-Streptomycin (Invitrogen, 100x)
0.4 μg/ml Hydrocortisone Succinate (Wako)
2 nM 3,3′,5-Triiodo-L-thyronine sodium salt (MP BIOMEDICALS)
100 nM Cholera Toxin (Calbiochem)
2 mM L-Glutamine (Invitrogen)
0.5% Insulin Transferrin Selenium (GIBCO, 200x)
10 ng/ml Recombinant Human EGF (R&D Systems)

### 2.2. Preparation of Differentiation Induction System (Modified SDIA Method)

### (1) Culture on PA6 Cells

The human iPS cells on the MEF feeders were treated with 0.25% trypsin/EDTA to disrupt the iPS cell colonies. The cells were pipetted a few times to collect a cluster population of iPS cell colonies (not used as single cells). The obtained iPS cell colonies were incubated on a gelatin-coated culture dish in a differentiation medium containing 0.5 nM BMP4 for approximately 1 to 2 hours. The supernatant was collected to thereby allow only the MEF feeder cells to adhere to the dish and collect only the human iPS cells. The number of the obtained human iPS cell colonies was counted. The iPS cells were inoculated at a density of 100 to 1000 colonies/cm² onto a culture dish inoculated with PA6 cells. The iPS cells were cultured at 37° C. for 8 days in a differentiation medium shown below in a 5% CO₂ atmosphere and subsequently cultured at 37° C. for 7 to 22 days in an epithelial induction medium. The cells were appropriately fixed with PFA. Furthermore, difference was also evaluated between the presence and absence of addition of FBS to the epithelial induction medium.

Differentiation medium (modified SDIA method)

G-MEM (Invitrogen)
10% KSR (Invitrogen)
2 mM L-Glutamine (Invitrogen)
1% Pyruvate (Invitrogen, 100x)
1% nonessential amino acids (Invitrogen, 100x)
0.1% 2-mercaptoethanol (Invitrogen)
0.5 nM BMP-4 (R&D System)

Epithelial induction medium (modified SDIA method)

Differentiation medium (−10% KSR) +10% FBS (Japan bio serum)* *A differentiation medium except for KSR was supplemented with 10% FBS and used as an epithelial induction medium.

### (2) Culture on 3T3 Cells

Human iPS cell colonies prepared in the same way as in the preceding paragraph were inoculated at a density of 10 to 1000 colonies/cm² onto a culture dish inoculated with 3T3 cells treated with MMC as feeder cells. The iPS cells were cultured at 37° C. for 8 days in a differentiation medium in a 5% CO₂ atmosphere and subsequently cultured at 37° C. for 7 to 22 days in an epithelial induction medium. The cells were appropriately fixed with PFA. Furthermore, difference was also evaluated between the presence and absence of addition of FBS to the epithelial induction medium.

3. Verification of Cells into Which Differentiation had Been Induced

The cells after differentiation induction were analyzed by an immunostaining method in the same way as in Example 1.

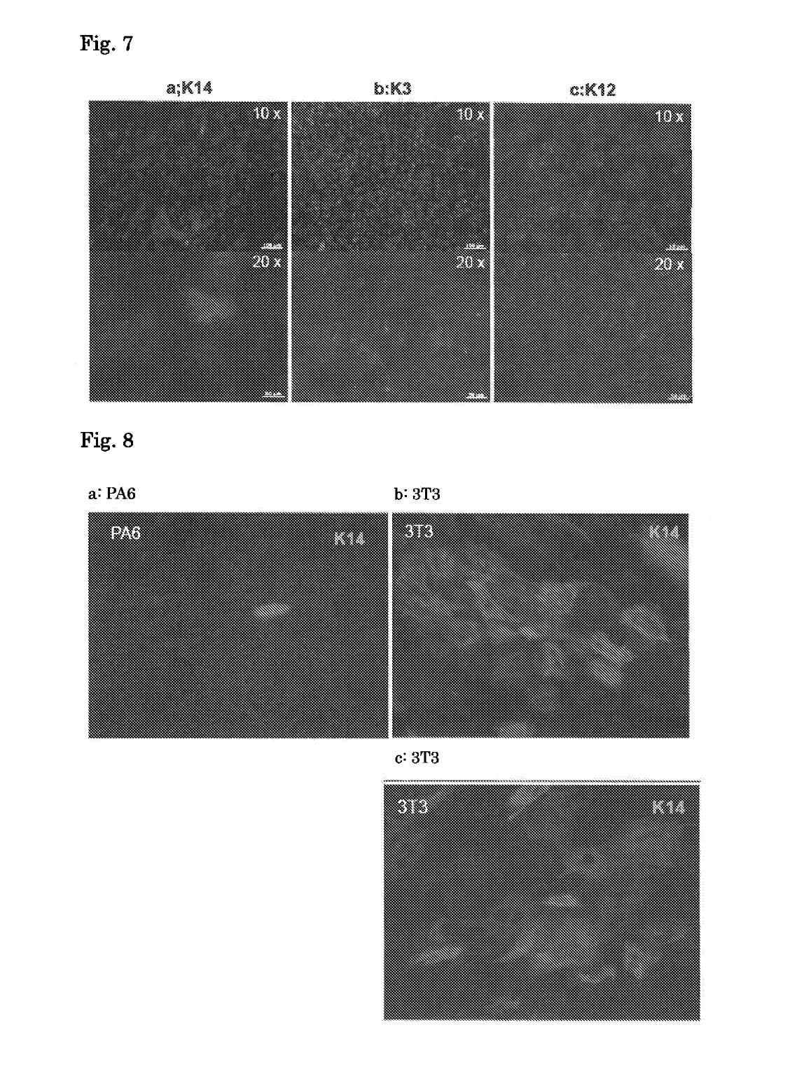

As a result, it was shown that keratin 14-positive epithelial progenitor cells, and keratin 12-positive corneal epithelial cells can be induced from human iPS cells at day 15 using the modified KCM method (FIG. 7). Moreover, in the modified SDIA method, keratin 14positive epithelial progenitor cells were hardly observed at day 15 when PA6 cells were used as feeders, whereas many keratin 14-positive cells were observed when 3T3 feeder cells were used as feeders (FIG. 8).

### Example 3

### Effect of Retinoic Acid on Induction of Differentiation into Epithelial Cells (Modified KCM Method)

The modified KCM method shown in Example 1 was examined for the influence of retinoic acid addition on the induction efficiency of differentiation into epithelial cells from mouse iPS cells or ES cells.

### 1. Differentiation Induction in Presence of Retinoic Acid

### (1) Immunostaining Method

According to Example 1, mouse iPS cells were cultured on collagen using (i) a KCM medium, (ii) a KCM medium supplemented with 0.5 nM BMP4, or (iii) a KCM medium supplemented with 0.5 nM BMP4+1 μM retinoic acid.

Likewise, mouse ES cells (RF8; provided by Dr. Robert Farese, Jr. from the Gladstone Institute) were cultured on collagen using (i) a KCM medium, (ii) a KCM medium supplemented with 0.5 nM BMP4, or (iii) a KCM medium supplemented with 0.5 nM BMP4+1 μM retinoic acid.

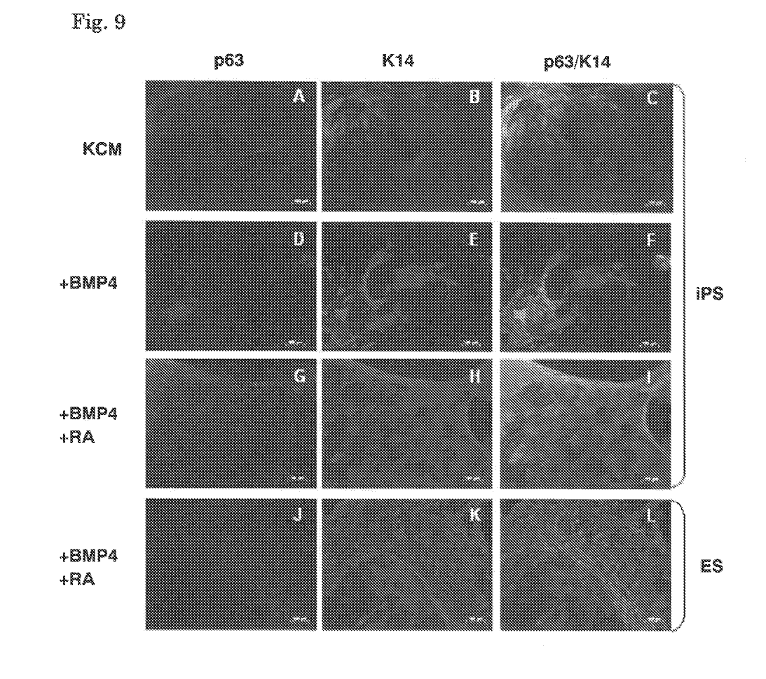

The cells after 21-day culture (differentiation induction) were examined for their respective expressions of p63 (red) and keratin 14 (K14: green) by an immunostaining method. The results are shown in FIG. 9 (FIGS. 9A to 9C: mouse iPS (KCM), FIGS. 9D to 9F: mouse iPS (KCM+BMP), FIGS. 9G to 9I: mouse iPS (KCM+BMP+retinoic acid), and FIGS. 9J to 9L: mouse ES (KCM+BMP+retinoic acid)).

As shown in FIG. 9, it was confirmed that high expressions of the epithelial cell markers p63 and keratin 14 (K14) were observed in both the iPS cells and the ES cells by the addition of 1 μM retinoic acid.

### (2) Real-Time PCR

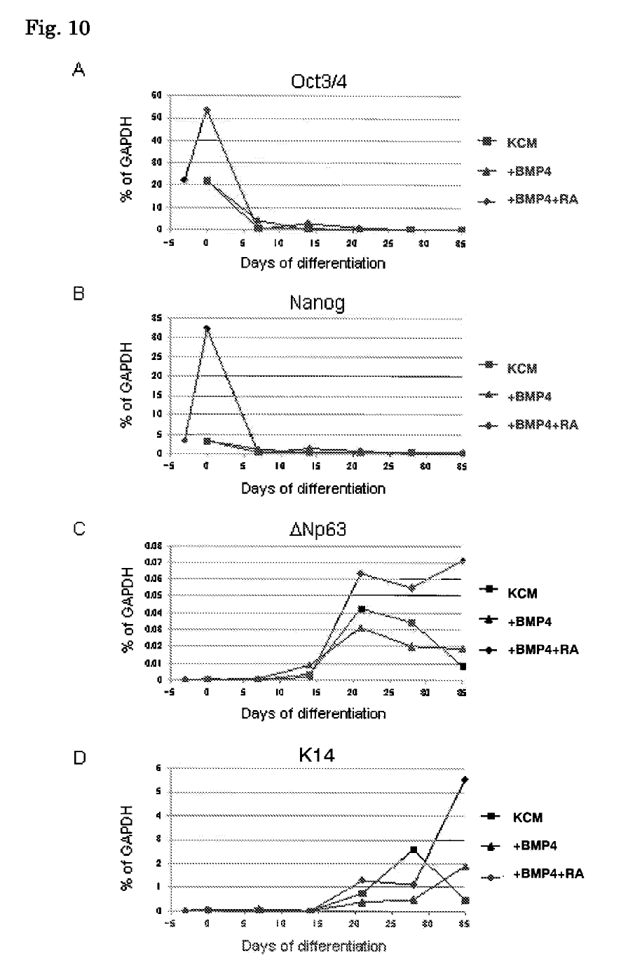

Mouse iPS cells and ES cells were separately cultured on collagen using (i) a KCM medium, (ii) a KCM medium supplemented with 0.5 nM BMP4, or (iii) a KCM medium supplemented with 0.5 nM BMP4+1 μM retinoic acid in the same way as in the preceding paragraph. The expressions of Oct3/4, Nanog, ΔNp63, and keratin 14 (K14) were quantified by real-time PCR at each day of induction. The results are shown in FIG. 10 (FIG. 10A: Oct3/4, FIG. 10B: Nanog, FIG. 10C: ΔNp63, and FIG. 10D: keratin 14 (K14)). In the diagram, before day 0 for the retinoic acid-supplemented groups, usual culture was performed on SNL feeders after addition of retinoic acid (see Example 1).

As shown in FIG. 10, the ES cell markers Oct3/4 and Nanog almost disappeared at day 7 or later by any of the differentiation induction methods. On the other hand, the expressions of the epithelial progenitor cell markers ΔNp63 and K14 increased from day 7 onward, and their expression levels exhibited the highest tendency by the addition of BMP4 and retinoic acid.

### 3. Discussion

These results demonstrated that the addition of retinoic acid remarkably improved the induction of differentiation into epithelial cells from iPS cells or ES cells by the modified KCM method.

### Example 4

### Effect of Retinoic Acid on Induction of Differentiation into Epithelial Cells (Modified SDIA Method)

Retinoic acid was added to the differentiation medium shown in Example 2 and examined for its influence on the induction of differentiation into epithelial cells from human iPS cells.

The present inventors used a KCM medium here, because it was confirmed as to human iPS cells that use of the KCM medium improved induction efficiency.

1. Differentiation induction in presence of retinoic acid

According to Example 2, human iPS cells were inoculated as a cell mass onto 3T3 or PA6 feeders and cultured in a differentiation medium supplemented with 0.5 nM BMP4 and 1 μM retinoic acid. Then, the medium was replaced by a KCM medium at day 8, and the cells were cultured for 2 to 8 weeks with the medium replaced every other day.

The outline of the culture method will be shown below. [Formula 1]

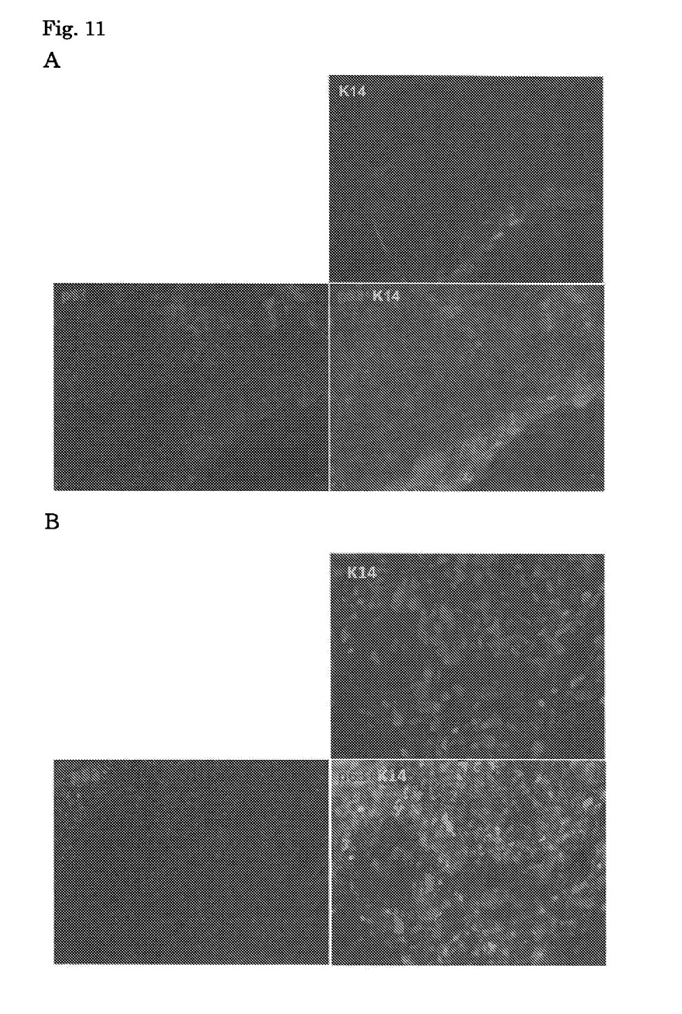

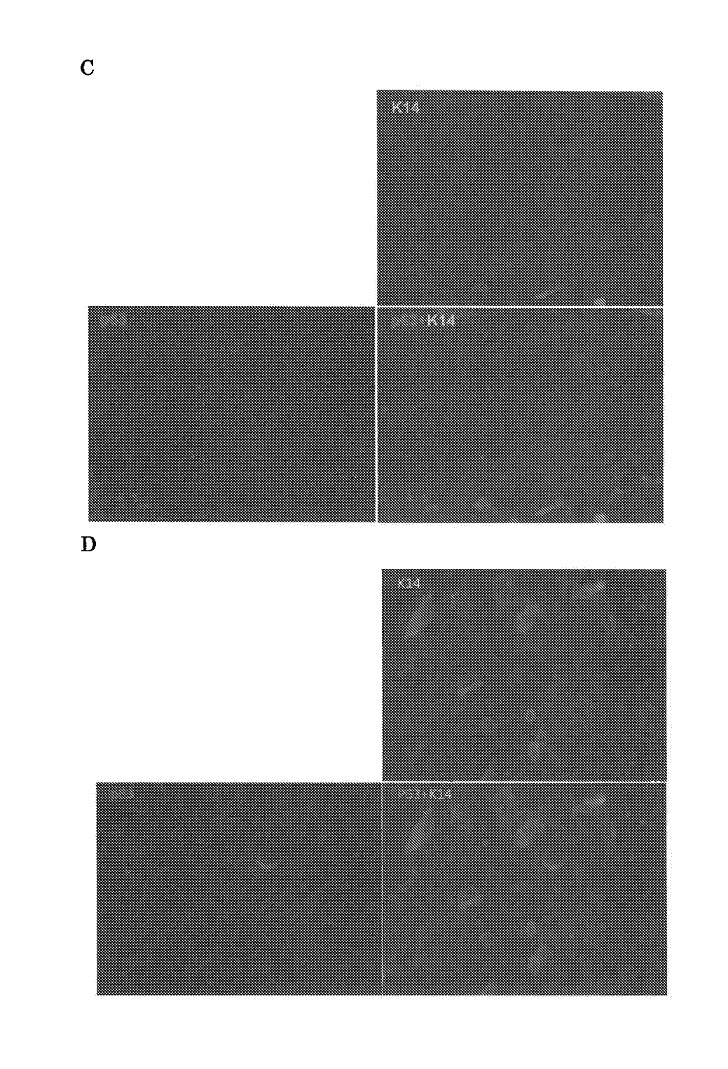

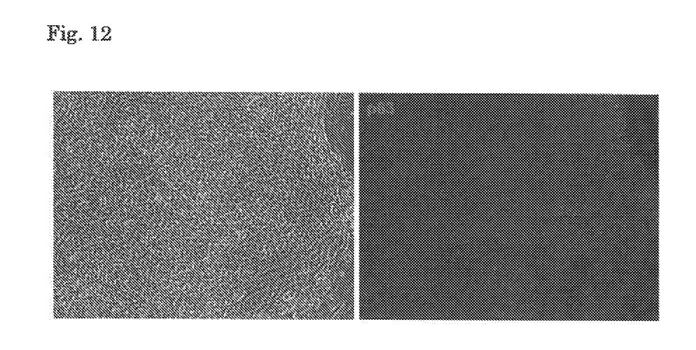

The cells after culture were examined for their respective expressions of p63 (red) and keratin 14 (K14: green) by an immunostaining method. Results of 15-day (differentiation medium for 8 days+KCM medium for 7 days) and 29-day (differentiation medium for 8 days+KCM medium for 21 days) culture on 3T3 feeders using a differentiation medium supplemented with 0.5 nM BMP4 and 1 μM retinoic acid and a KCM medium, results of 15-day culture using a differentiation medium supplemented with 0.5 nM BMP4 without the addition of retinoic acid and a KCM medium, and results of 15-day (differentiation medium for 8 days+epithelial induction medium for 7 days) culture on 3T3 feeders using a differentiation medium supplemented with 0.5 nM BMP4 and 1 μM retinoic acid and an epithelial induction medium are shown in FIGS. 11A to 11D, respectively. Moreover, results of 15-day (differentiation medium for 8 days+KCM medium for 7 days) culture on PA6 feeders using a differentiation medium supplemented with 1 μM retinoic acid are shown in FIG. 12.

In the culture on the 3T3 feeders, high expression of the epithelial cell marker p63 was observed at day 15 (FIG. 11A), and the expression of K14 subsequent to the p63 expression was also confirmed (FIG. 11B). By contrast, when no retinoic acid was added, p63-positive cells did not appear even at day 15 (FIG. 11C), and p63-positive and K14-positive cells hardly appeared at days subsequent to day 15. When retinoic acid was added on the PA6 feeders, high expression of the epithelial cell marker p63 was observed at day 15 as in the case using 3T3 feeders (FIG. 12). Moreover, in the differentiation medium+epithelial induction medium usually used in the SDIA method, p63-positive cells did not appear at day 15 even by the addition of retinoic acid (FIG. 11D).

### 3. Discussion

These results demonstrated that also in the modified SDIA method, the addition of retinoic acid was useful for the induction of differentiation into epithelial cells. It was also confirmed that more excellent induction efficiency of differentiation into epithelial cells from human iPS cells was obtained using the differentiation medium+KCM medium than using the differentiation medium +epithelial induction medium usually used in the SDIA method.

### INDUSTRIAL APPLICABILITY

The present invention is free from concerns about donor shortage and rejection. The present invention is useful as novel regenerative medicine for corneal epithelial disease. Furthermore, epidermal cells or various epithelial layers such as and oral mucosal epithelium can be regenerated using the epithelial stem cells/progenitor cells of the present invention as a cell source. Specifically, the present invention is applicable as a basic technique for autologous regenerative medicine techniques for various epithelial diseases. Furthermore, an epithelial cell bank capable of reducing rejection can also be prepared by developing epithelial cells on a HLA genotype basis using the present invention.

All publications, patents, and patent applications cited herein are incorporated herein by reference in their entirety.

1. A method for inducing differentiation into a keratin 14-positive and p63-positive epithelial progenitor cell/stem cell population from induced pluripotent stem cells induced from mammalian somatic cells or undifferentiated stem cells, comprising:
culturing said induced pluripotent stem cells on feeder cells or a support selected from collagen, basement membrane matrix, amnion, fibronectin, and laminin using a medium for epidermal cells containing an epidermal growth factor and/or cholera toxin and serum.

2. The method according to claim 1, wherein the medium further contains one or more selected from hydrocortisone, insulin, transferrin, and selenium.

3. The method according to claim 1, wherein the feeder cells are stromal cells.

4. The method according to claim 3, wherein the stromal cells are 3T3 cells.

5. The method according to claim 1, wherein the medium further contains at least one of BMP4 (Bone Morphogenetic Protein 4) retinoic acid.

6. The method according to claim 1, wherein the differentiation into the epithelial progenitor cell/stem cell population is induced without embryoid body formation.

7. A method for inducing differentiation into a keratin 14-positive and p63-positive epithelial progenitor cell/stem cell population from induced pluripotent stem cells induced from mammalian somatic cells or undifferentiated stem cells, comprising:
culturing said induced pluripotent stem cells on 3T3 cells or in the presence of a 3T3 cell-derived differentiation factor.

8. The method according to claim 7, wherein the induced pluripotent stem cells are cultured in an epithelial induction medium containing serum and/or BMP4 or in a medium for epidermal cells containing an epidermal growth factor and/or cholera toxin and serum.

9. The method according to claim 8, wherein the epithelial induction medium further contains one or more selected from retinoic acid, nonessential amino acid, β-mercaptoethanol, and sodium pyruvate, and the medium for epidermal cells further contains one or more selected from hydrocortisone, insulin, transferrin, and selenium.

10. The method according to claim 8, wherein the induced pluripotent stem cells are cultured in a differentiation medium containing one or more selected from a serum substitute, BMP4 (Bone Morphogenetic Protein 4), and retinoic acid and then further cultured in the epithelial induction medium or the medium for epidermal cells.

11. The method according to claim 10, wherein the differentiation medium further contains one or more selected from nonessential amino acid, β-mercaptoethanol, and sodium pyruvate.

12. A method for inducing further differentiation into an epithelial cell population from the epithelial progenitor cell/stem cell population which has been induced by a method according to claim 1.

13. The method according to claim 12, wherein the epithelial cell population is any selected from a corneal epithelial cell population, an oral mucosal epithelial cell population, a urinary bladder epithelial cell population, a conjunctival epithelial cell population, a gastric mucosal epithelial cell population, a small intestinal epithelial cell population, a large intestinal epithelial cell population, a renal epithelial cell population, a renal tubular epithelial cell population, a gingival mucosal epithelial cell population, an esophagus epithelial cell population, a hepatic epithelial cell population, a pancreatic epithelial cell population, a pulmonary epithelial cell population, and a gallbladder epithelial cell population.

14. A method for inducing differentiation into a keratin 12-positive corneal epithelial cell population from the epithelial progenitor cell/stem cell population, comprising continuing to culture in a method according to claim 1.

15. The method according to claim 1, further comprising the step of isolating a keratin 14-positive and p63-positive cell population.

16. The method according to any one of claim 14, further comprising the step of isolating a keratin 12-positive and keratin 14-negative cell population.

17. A cell preparation for epithelial disease comprising an epithelial progenitor cell/stem cell population obtained by a method according to claim 1 and/or an epithelial cell population induced from said epithelial progenitor cell/stem cell population.

18. A cell sheet comprising layers of an epithelial progenitor cell/stem cell population obtained by a method according to claim 1 and/or an epithelial cell population induced from said epithelial progenitor cell/stem cell population.

19. (canceled)

20. (canceled)

21. (canceled)

22. (canceled)

23. (canceled)

24. (canceled)
